(19) United States
(12) Patent Application Publication
(10) Pub. No.: US 2004/0102819 A1
(43) Pub. Date: May 27, 2004

(54) Portable therapeutic device and method for pain relief

(76) Inventors: Shazhou Zou, Glenwood, MD (US); Victor W. Wang, Herndon, VA (US); Tianxin Wang, Columbia, MD (US)

Correspondence Address: Tianxin Wang, 9768 Early Spring Way, Columbia, MD, 21046, US

(21) Appl. No.: 10/716,352

(22) Filed: Nov. 18, 2003

Related U.S. Application Data
(60) Provisional application No. 60/427,827, filed on Nov. 20, 2002.

(51) Int. Cl. A61N 1/18

(52) U.S. Cl. 607/46

(57) ABSTRACT

The invention relates to a method and device of reducing pain and other symptoms in a patient utilizing a non-invasive electrical stimulation device applied over acupuncture points and adjacent nerves of a patient. The non-invasive electrical stimulation device is a portable electrical therapeutic device. The device includes electronic circuitry for imparting electrical impulses to the desired acupuncture points and areas. Batteries within the device power the electric circuitry, and an operable switch on the device controls on/off and pulse frequency and amplitude.

The present application claims priority under 35 U.S.C. § 119(e) to U.S. Provisional Application No. 60/427,827, filed on Nov. 20, 2002, which provisional application is hereby incorporated by reference in its entirety.

### BACKGROUND OF THE INVENTION

1. Field of the Invention

This invention relates to a portable therapeutic device and a non-chemical and non invasive means for reducing and alleviating pain. The invention utilizes electrical current passed through acupuncture sites on the human body for the control of pain.

2. Background Information

Although there are many effective pain relief medications available today, there are also drawbacks associated with the use of chemical compositions. Many pain relief drugs have serious side effects such as addiction, vomiting and stomach irritation. Many people are also allergic to certain chemical medications and therefore cannot use this form of treatment to alleviate pain. Around one third of the world populations suffer from headache. Hundreds of millions suffer from varieties of pains such as injury pain, cancer pain, surgical pain, toothache and arthritis pain. Consequently, the need to relieve pain by means other than chemical medications is highly desired. Acupuncture is a traditional Chinese therapeutic technique involving the stimulation of certain areas or nerves and nerve junctions by puncturing the skin with fine needles to produce a certain physiological effect. It has effectively been in use for many centuries in China for pain relief and other medical purposes. Although it has found that Chinese acupuncture techniques can stimulate the production of endogenous opioid peptides, which have analgesic effects, the exact means by which this is accomplished is still unclear. However, the results are conclusive. Acupuncture were even used as alternative anesthesia for surgery.

A patent search was conducted to examine non-chemical means for reducing and alleviating pain. The following prior art patents were located in the course of the patent search, and are considered to be the references most pertinent to the invention.

The Wilson U.S. Pat. No. 4,398,545, issued on Aug. 16, 1983, illustrates a device designed to block pain impulses originating from an injury;

The Hoffmann U.S. Pat. No. 3,107,672, and the Moss U.S. Pat. No. 3,424,165, are directed toward stimulating and firming the muscles for cosmetic purposes;

The Symmes U.S. Pat. No. 3,889,163, the Fischell U.S. Pat. No. 4,440,160, and the Crossley U.S. Pat. No. 4,715,367, teach devices which assist in refraining from undesirable habits. These devices utilize painful or alarming electrical stimulation.

The Batters U.S. Pat. No. 4,664,118, issued on May 12, 1987, illustrates a device designed to reduce pain and edema in a human body using an electrically conducting material that envelopes hand, foot and ankle to apply an electrical shock to the injured part.

The Bertolucci U.S. Pat. No. 4,981,146, issued on Jan. 1, 1991, illustrates a nausea control electrical stimulating device in the form of a watch-like housing and related attachment band for mounting onto the human wrist.

The Gruzdowich U.S. Pat. No. 6,393,324, issued on May 21, 2002, illustrates a blood pressure moderating device same to the device used in the above Bertolucci patent.

None of the prior art patents examined are specifically structured as fully self-contained, portable and wearable devices for reducing or eliminating pain. Some of the prior art devices are directed toward stimulating a general area of the body or a muscle or muscle group and are not designed to stimulate specific acupuncture points or nerves to alleviate pain. Other devices examined are designed to produce an irritating or even painful electric impulse to produce a certain desired response in the person wearing the device. Several of the prior art devices require administration by one experienced and skilled with the equipment, whereas our device is simple and easy to use, requiring no special training or experience.

The device in our invention is primarily directed towards stimulating one or more of the acupuncture points selected from NeiGuan, WaiGuan, LieQue and HeGu located in the human wrist and hand for the purpose of alleviating pain. The invention is completely portable and self-contained. It can also be safely and painlessly self-administered.

### SUMMARY OF THE INVENTION

The methods and devices described below use electro-acupuncture applied to acupuncture points on the wrist and hand to control pain. A patient desiring to moderate his or her pain places the electrodes on the acupuncture points selected from NeiGuan, WaiGuan, LieQue and HeGu, and applies electrical stimulation through these electrodes. This has the effect of reducing symptoms of pain, such as headaches, post surgery pain, injury pain, cancer pain, toothache and arthritis pain. The pulse generating circuitry, and power supply are most conveniently packaged in a housing which is held to the forearm or wrist or hands with a band or a structure having similar function (such as a bangle or bracelet like structure or a rigid/semirigid closure or a U shape clamp like structure) or with adhesive on the device. One or more external electrodes are linked with the housing. The device is placed so that the electrode/electrodes overlie the acupuncture points known to effect a desired therapy. Alternatively, the device is placed so that the electrodes overlie a nerve that runs under the acupuncture point, in which case the device may be placed some distance from the associated acupuncture point to provide a comfortable placement for the device.

### BRIEF DESCRIPTION OF THE DRAWINGS

FIG. 1 shows the position of preferred acupuncture points to relieve pain.

FIG. 2 is a perspective illustration of one embodiment of the device in the invention as worn on the wrist of a person using the device to suppress pain.

FIG. 3 is a perspective illustration of one embodiment of the device in the invention as worn on the forearm of a person using the device.

FIG. 4 shows one embodiment of a block diagram of the electrical circuitry suitable for the invention

FIG. 5 is a perspective view of one preferred embodiment of the device

FIG. 6 is a bottom plan view of one embodiment of the device

FIG. 7 shows one embodiment of the external electrode assembly

FIG. 8 is a perspective illustration of one embodiment of the device in the invention as worn on the hand of a person using the device.

### DESCRIPTION OF THE INVENTIONS AND THE PREFERRED EMBODIMENT

The methods and devices described below use electro-acupuncture applied to acupuncture points on the wrist and hand to control pain. A patient desiring to moderate his or her pain places the device on his or her wrist or forearm and attaches the electrodes on the selected acupuncture points to apply electrical stimulation to these points through these electrodes. This has the effect of reducing pain caused by varieties of diseases. The primary acupuncture point to relive pain is HeGu. Preferred adjunctive-acupuncture points to relive pain can be selected from NeiGuan, WaiGuan and LieQue.

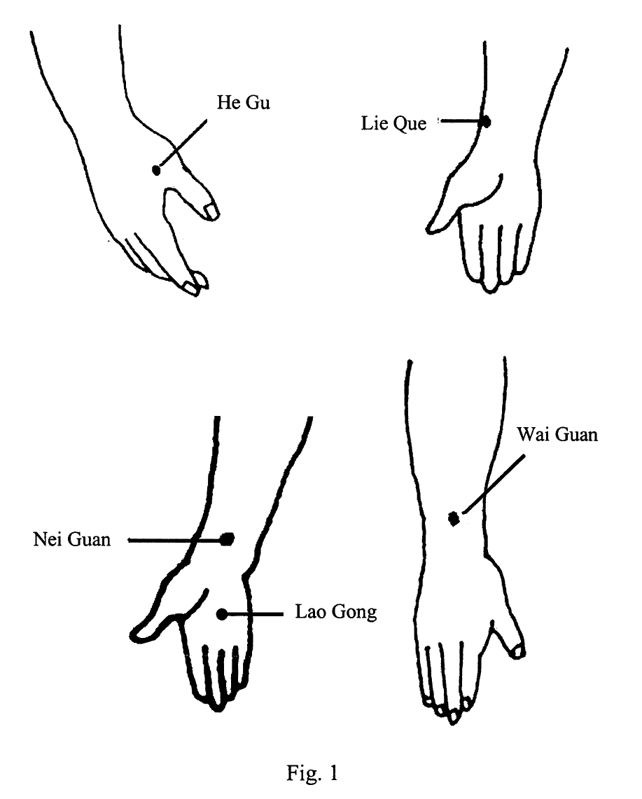

Using adjunctive acupuncture points in combination with HeGu gives higher pain relieving effect than using these adjunctive acupuncture points alone. The position of these acupuncture points are illustrated in FIG. 1. Stimulating other acupuncture points selected from LaoGong, TaiYang, ShaoShang, ZuSanLi, FengLong, GuanChong, ShangYang, YeMen, SiZhuKong, YangBai, FengChi, SanYinjiao, YinLingQuan and YangLingQuan also shows certain pain relieving effect, but not as effective as stimulating HeGu or HeGu plus adjunctive acupuncture points. In addition, stimulating some of these points requires attaching the device of the invention in places other than the wrist or arm. The preferred electrical pulse repetition rate of approximately 2 pulses per second with a pulse width of 600 microseconds, or approximately 15 pulses per second with a pulse width of 300 microseconds, or approximately 100 pulses per second with a pulse width of 200 microseconds, or the combination of the above wave patterns has been found to provide effective relief of pain in patients. The preferred electrical power levels are about 2-50 milli-amps peak pulse height. A wider range of pulse patterns can also be used in the non-invasive nerve stimulation devices. Bi-directional pulse is preferred to avoid skin damage. We had tested our device in patients suffering varieties of pains including headaches, post surgery pain, injury pain, toothache and arthritis pain and found that most patients feel significant relief of pain within 15 minutes of using our device. It also significant reduces the symptom of the people suffering nausea when the stimulating points include one of more acupuncture points selected from NeiGuan, GongSun, SanYinJIao and YinLingQuan.

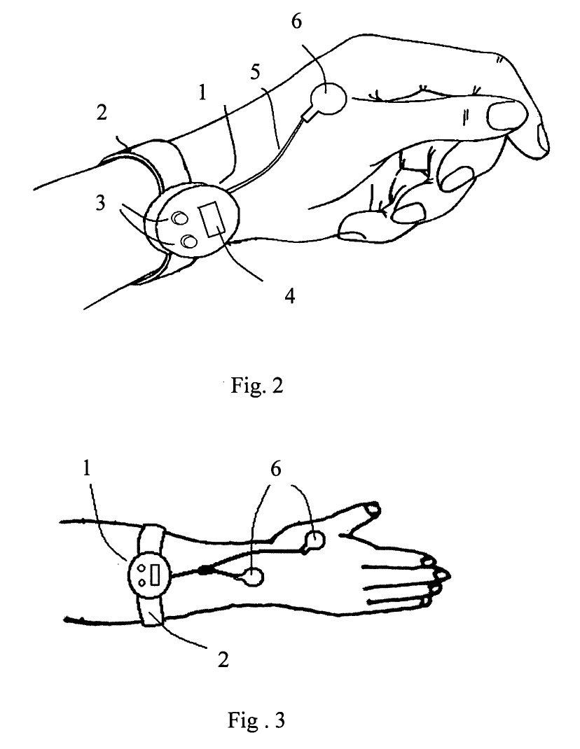

FIG. 2 is a perspective illustration of one embodiment of the device of the invention as worn on the wrist of a person using the device to suppress pain. The power supply and pulse generating circuitry for the invention are located within a watch-like or small box like housing 1. Housing 1 may be manufactured in a multitude of shapes, preferably be sized similar to that of a wristwatch housing to allow placement against a human wrist or arm. In the drawings, housing 1 is shown as an oval structure having two stimulating level control buttons 3 and one LCD display 4 on its top surface. The two power level control buttons 3 control the output power level of the electrodes. Higher power level (the intensity of the stimulation current) generally give better therapeutic effects. The patients use the control buttons to adjust the power level to achieve desired therapeutic effects and the best comfort. These buttons may also be used to control the pulse patterns. The LCD display 4 displays the working status of the device such as the current power level and pulse pattern. A timing function can also be incorporated within the LCD. The adjustable attachment band 2 fastens the housing 1 to the wrist. The attachment band 2 can also be used to mount the housing 1 onto the human ankle or calf, legs, head, arms and other areas. An external electrode assembly 6 is attached to the HeGu point. The housing 1 sends out electrical pulse via wire 5 to the external electrode assembly 6 to apply electrical stimulation to the patients. The external electrode assembly 6 could contain one or more electrodes on the surface of the electrode assembly that contacts the human skin. If there is only one electrode on the external electrode assembly 6 and only one external electrode assembly 6 is linked with housing 1, the back surface of housing 1 that contacts the skin or the band 2 could be made of electrical conductive material to function as the second electrode to allow the electrical current flow. In the embodiment in the drawing, the external electrode assembly 6 has only one electrode and the bottom of the housing 1 has another electrode 8 (details described in FIG. 6). The electrode 8 could be mounted either on NeiGuan or WaiGuan position or other position; in the drawing, it is attached to the NeiGuan point, therefore the electrical pulse can be applied to both NeiGuan and HeGu simultaneously. If the external electrode assembly 6 contains only one electrode, it is essentially an external electrode with a link to the housing 1. If two electrodes are assembled on the external electrode assembly 6, the electrodes can be mounted in the fixed arrangement such as side by side on the skin-contacting surface of the external electrode assembly 6. The current flow can provide stimulation to a point between the two electrodes therefore avoiding the need of using band 2 or the bottom of housing 1 as the second electrode. Self-adhesive electrodes could be used in external electrode assembly 6. A rigid or semi rigid connection structure can be used to link the external electrode assembly 6 with the housing 1 besides using wires.

FIG. 3 is an illustration of one embodiment of the device in the invention as worn on the forearm of a person using the device. Two external electrode assembly 6 are linked to the housing 1 and attached to HeGu and WaiGuan points to apply pulse to both points simultaneously. Using more external electrode assembly, it is possible to stimulate more points simultaneously.

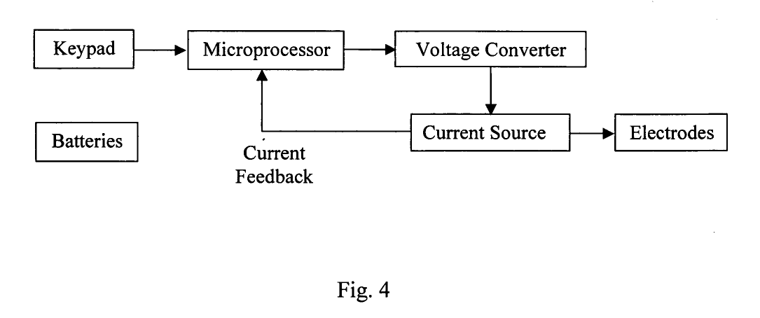

The electrical circuitry of the device can be implemented with well-known art. There are many designs to implement the circuitry such as those listed in U.S. Pat. No. 4,981,146. FIG. 4 illustrates a example of the circuit diagram suitable for the device. The microprocessor receives input from the keypad to set the intensity level of the stimulate pulses, and current feedback to regulate the microprocessor output to voltage converter and current source to generate the stimulate pulses at the current level set by the keypad. The voltage converter converts battery voltage to high voltage. The output of the current source is connected to the electrodes, which contact skin. The batteries supply power to every block. An external power source can also be connected to the housing 1 to provide longer operation time.

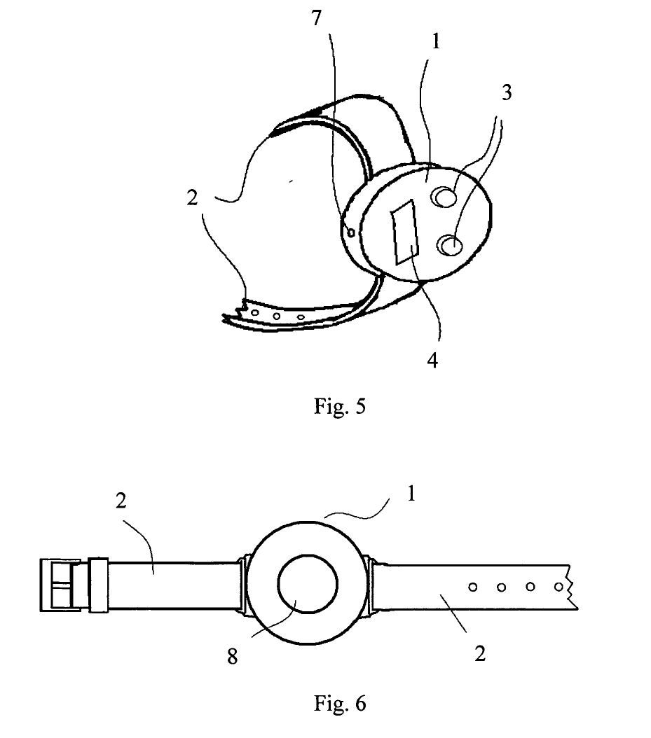

FIG. 5 is a perspective view of one preferred embodiment of the device. Socket 7 is used to link the external electrode assembly 6.

FIG. 6 is a bottom plan view of one embodiment of the device. Electrode 8 is assembled on the bottom surface of housing 1.

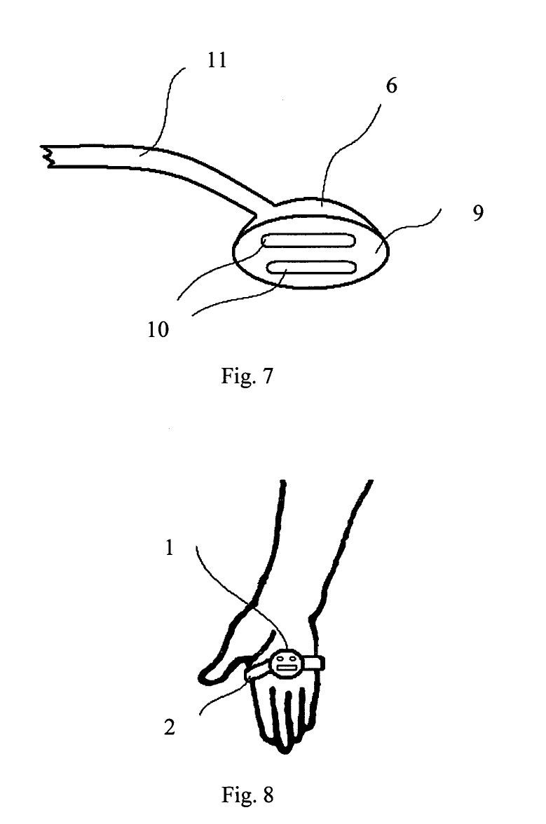

FIG. 7 shows one embodiment of the external electrode assembly. On the skin-contacting surface 9 of the external electrode assembly 6, two electrodes 10 are aligned in a side by side manner. The external electrode assembly 6 is connected with the housing 1 via connection means 11. Here the connection means 11 is a semi rigid bridge like structure that can be bent to allow the fully contact of electrodes 10 with the skin. Electrical wires inside the connection means 11 are linked with housing 1 to deliver the electrical pulse to the electrodes 10.

The device in the invention can also be attached to positions other than arm and wrist. FIG. 8 illustrates of one embodiment of the device in the invention as worn on the hand of a person using the device. The housing 1 is attached to the palm via band 2. The electrodes at the back of housing 1 contact the LaoGong point. An external electrode assembly can be linked with housing one to stimulate the HeGu at the back of hand of other stimulation points.

What is claimed is:

1. A portable therapeutic device for achieving certain therapeutic effects by electrical stimulation of the acupuncture points at a human body, said device comprising:
a small housing having a size and shape adapted to be worn on the human wrists or arms or hands or legs;
at least one external electrode assembly that can be linked with said housing;
circuit means mounted within said housing, said circuit means having means for delivering pulsed electrical stimulation signals of selected amplitude and cycle rate to said external electrode assembly;
band or structure or adhesive means connected to said housing and adapted to be fastened about the human wrists or arms or hands or legs;

2. The device of claim 1 wherein said stimulation signals are pulsed electrical current signals.

3. The external electrode assembly of claim 1 is linked with the said housing via wires or rigid or semi rigid connection means.

4. The external electrode assembly of claim 1 has one or more electrodes on its surface that contact human body.

5. The certain therapeutic effects of claim 1 are pain relief and nausea control.

6. The acupuncture points of claim 1 for pain relieving are selected from NeiGuan, WaiGuan, LieQue and HeGu.

7. The pulsed electrical current signal in claim 2 has cycle rates between 0.1 to 1000 pulses per second.

8. The device of claim 2 wherein said circuit means includes means for variably adjusting the pattern and amplitude of said stimulation signals.

9. The device of claim 2 wherein said circuit means further includes an on/off switch.

10. The device of claim 1 has electrical conducting surface on the back of the said housing or said band or said structure means.

11. The electrical conducting surface in claim 10 can be mounted onto the acupuncture point to apply electrical stimulation.

12. The electrical conducting surface of claim 11 is electrode.

13. A method of controlling pain, comprising the steps of:
mounting a pair of electrodes or at least one electrode at a position generally closely overlying HeGu position; and
generating pulsed stimulation signals of selected amplitude, pulse width and cycle rate; and
delivering the stimulation signals to the said electrodes to stimulate the selected acupuncture points and adjacent nerves to relieve pain.

14. A method of controlling pain, comprising the steps of:
fasten the device in claim 1 to wrist or arm or hands; and
mounting one or more external electrode assembly at a position generally closely overlying one or more acupuncture points selected from NeiGuan, WaiGuan, LieQue and HeGu; and
generating pulsed stimulation signals of selected amplitude, pulse width and cycle rate; and
delivering the stimulation signals to external electrode assembly to stimulate the selected acupuncture points and adjacent nerves to relieve pain.

15. A method of controlling pain, comprising the steps of:
fasten the device in claim 10 to wrist; and
mounting one or more external electrode assembly at a position generally closely overlying one or more acupuncture points selected from NeiGuan, WaiGuan, LieQue and HeGu; and
mounting the said electrical conducting surface at a position generally closely overlying one or more acupuncture points selected from NeiGuan, WaiGuan and LieQue; and
generating pulsed stimulation signals of selected amplitude, pulse width and cycle rate; and
delivering the stimulation signals to said external electrode assembly and the said electrical conducting surface to stimulate the selected acupuncture points and adjacent nerves to relieve pain.

16. The method of claim 13 further including the step of adjustably varying the pattern and amplitude of the stimulation signals.

17. The method of claim 14 further including the step of adjustably varying the pattern and amplitude of the stimulation signals.

18. The method of claim 15 further including the step of adjustably varying the pattern and amplitude of the stimulation signals.
